(19) United States
(12) Patent Application Publication
(10) Pub. No.: US 2004/0006052 A1
(43) Pub. Date: Jan. 8, 2004

(54) Injection solutions to treat neuropathic symptoms and other sensorimotor disorders that are due to neuromas of the foot or other entrapment neuropathies of peripheral nerves, and method of use

(76) Inventors: Charles J. Gudas, Charleston, SC (US); Ludwig A. Lettau, Mt. Pleasant, SC (US)

Correspondence Address: ROBERT J. DEPKE LEWIS T. STEADMAN, HOLLAND & KNIGHT LLC, 131 SOUTH DEARBORN, 30TH FLOOR, CHICAGO, IL, 60603, US

(21) Appl. No.: 10/412,933

(22) Filed: Apr. 14, 2003

Related U.S. Application Data
(60) Provisional application No. 60/372,590, filed on Apr. 15, 2002.

(51) Int. Cl. A61K 31/573; A61K 31/445; A61K 31/137

(52) U.S. Cl. 514/171; 514/317; 514/649; 514/537

(57) ABSTRACT

Injection solutions to treat nerve disorders of the foot are disclosed. A method of treatment is also disclosed

### This application claims domestic priority from U.S. Provisional Application, Serial No. 60/372,590, filed Apr. 15, 2002.

### BACKGROUND OF THE INVENTION

A Morton's neuroma of the foot is a focal enlargement of an interdigital nerve that most commonly occurs between the third and fourth metatarsal heads where the nerve stretches under the transverse metatarsal ligament. Chronic irritation of the interdigital nerve at that site results in inflammation and fibrotic thickening which over time results in the formation of an elongated lump called a neuroma. The pathogenesis and histopathology of a neuroma is consistent with an entrapment neuropathy. Neuromas may also form in the second interdigital nerve between the second and third metatarsal heads.

Neuromas are likely asymptomatic in the initial stages of development. The earliest symptoms are usually numbness of the middle toes or achy pains of the forefoot which are precipitated and worsened by tight shoes, high heels, or prolonged walking or standing. Symptoms may occur in only one foot or both feet with one foot worse, or both feet symmetrically. Symptoms tend to worsen over time and eventually involve both feet. As neuromas become more symptomatic, they mainly cause the neuropathic symptoms of burning/tingling pain, electric shock-like shooting pains, numbness and hypersensitivity.

Conservative initial management of neuromas may include the wearing of shoes with a wider toe box, padding, strapping, and biomechanical orthoses. Most patients eventually require interventional treatment in the form of neuroma injections or neuroma excisional surgery to adequately control symptoms.

Historically, the injection treatment of neuromas consisted of either local anesthetic with cortisone, or local anesthetic with absolute alcohol, injected into the internetatarsal neuroma-containg space in a series of 2 to 4 injections given about 2 weeks apart. The clinical success rates reported in the literature using either of these two injection solutions have been variable and inconsistent, and commonly excisional surgery has been necessary.

Because of the erratic effectiveness of the antecedent injection solutions, the inventors (Drs. Gudas and Lettau) devised a novel neuroma injection solution that is a novel and unique mixture composed of currently available medications and compounds. An injection series using this new mixture has consistently provided excellent symptomatic relief of the neuropathic symptoms of neuromas whether unilateral or bilateral. It is the opinion of the inventors that the effectiveness of the new injection mixture obviated the need for neuroma removal surgery in many patients. In addition injection of the new mixture has been found to be much better tolerated than the previously used solutions of either cortisone or absolute alcohol.

In a published study (Journal of the SC Medical Association 2002;98:221e-230e) by the inventors, it was determined that bilateral neuromas are common and are statistically associated with chronic neuropathic foot pain among patients with HIV infection and patients with diabetes. Treatment (using the new injection mixture) of the neuromas in these patients resulted in substantial improvement in, and in some cases complete resolution of, their neuropathic foot symptoms. The inventors, as a result of these investigations, believe that intermetatarsal neuromas are the major cause of chronic neuropathic foot pain and dysesthesias in patients with HIV or diabetes.

Additional experience of the inventors using the new injection mixture has shown it to be effective in treating the neuropathic foot symptoms of neuromas in a variety of patients who have neither HIV nor diabetes. Anecdotal experience of the inventors indicates that the new injection mixture may be useful in other entrapment neuropathies caused by keloid or other scar tissue that are associated with symptoms of neuropathic pain and dysesthesias, as well as restless legs syndrome and periodic leg movement disorder which may be associated with symptomatic neuromas in the feet.

### BRIEF SUMMARY OF THE INVENTION

The indications for this new injection mixture invented by Drs. Gudas and Lettau potentially include: 1) intermetatarsal (Morton's type) neuroma and its various foot/leg manifestations including but not limited to exercise/stress related aching painful feet, numbness, burning/tingling sensations, electric shock shooting pains, hypersensitivity, tightness/cramping, and other unpleasant paresthesias and dysesthesias, 2) other entrapment neuropathies caused by keloid or other scar tissue that is associated with neuropathic pain and dysesthesias, 3) restless legs syndrome and periodic leg movement disorder in its various forms and manifestations, 4) polyneuropathy or other neuritis of sensory nerves that is associated with various disease conditions or neurotoxic effect of medications, or that is unassociated with any particular medical condition (idiopathic occurrence).

The invention is embodied in a solution comprised of the following general formulation: methylprednisolone acetate sterile aqueous suspension (40-80 mg/cc., dexamethasone sodium phosphate 4 mg/cc., bupivacaine hydrochloride (0.5-0.75% with or without epinephrine), lidocaine hydrochloride (1-2% with or without epinephrine), dehydrated alcohol. These medications are mixed for solutions in the following percentages: 25-45% bupivacaine hydrochloride, 25-45% lidocaine hydrochloride, 4% methylprednisolone acetate sterile aqueous suspension, 2% dexamethasone sodium phosphate, and 4-50% dehydrated alcohol. The bupivacaine hydrochloride may be utilized alone or in conjunction with lidocaine hydrochloride with or without epinephrine. Lidocaine hydrochloride may be utilized alone or in conjunction with bupivacaine hydrochloride with or without epinephrine.

The medication was utilized with and without preservatives and was injected in single and multiple dose injections for the treatment of symptomatic Morton's neuromas. The percentage of dehydrated alcohol may be increased to 50% of the total solution with a subsequent equal decrease in the amount of bupivacaine hydrochloride and lidocaine, or subsequent decrease in the amount of bupivacaine hydrochloride without the use of lidocaine hydrochloride, or decrease in the amount of lidocaine hydrochloride without the bupivacaine hydrochloride.

The specific examples of the solution that has been utilized are listed below.

GLAL Solution #1

45 ccs—0.5-0.75% (bupivacaine hydrochloride)

2 ccs—methylpredinisolone acetate sterile solution

1 cc dexamethasone phosphate/4 mg/cc

2 ccs absolute alcohol

50 ccs—total

GLAL Solution #2

45 ccs—2% lidocaine hydrochloride

2 ccs—methylpredinisolone acetate sterile solution—40 mg/cc

1 cc dexamehtazone phosphate/4 mg/cc

2 ccs absolute alcohol

50 ccs-total

GLAL Solution #3

45 ccs—0.5-0.75% bupivacaine hydrochloride with epinephrine

2 ccs methylpredinisolone acetate sterile solution—80 mg/cc

1 cc dexamethasone phosphate/4 mg/cc

2 ccs absolute alcohol

50 ccs total

GLAL Solution #4

45 ccs—2% lidocaine hydrochloride with epinephrine

2 ccs—methylpredinisolone acetate sterile solution—80 mg/cc

1 cc dexamenthasone phosphate/4 mg/cc

2 cc absolute alcohol

50 ccs—total

GLAS Solution #5

22.5 ccs—2% lidocaine hydrochloride mixed with 22.5 ccs 0.5% bupivacaine

hydrochloride

2 ccs—methylprednisolone acetate sterile solution—80 mg/cc

1 cc dexamenthasone phosphate/4 mg/cc

2 cc absolute alcohol

50 ccs—total

GLAL Solution #6

22.5 ccs—1% lidocaine hydrochloride mixed with 22.5 ccs 0.5% bupivacaine

hydrochloride

2 ccs methylprednisolone acetate sterile solution—40 mg/cc

1 cc dexamenthasone phosphate/4 mg/cc

2 cc absolute alcohol

50 ccs—total hydrochloride

GLAL solution #7

22.5 ccs 1% lidocaine hydrochloride with epinephrine mixed with 22.5 ccs

0% bupivacaine hydrochloride with epinephrine

2 ccs methylprednisolone acetate sterile solution—40 mg/cc

1 cc dexamenthasone phosphate/4 mg/cc

2 cc absolute alcohol

50 ccs—total

GLAL solution #8

22.5 ccs 2% lidocaine hydrochloride with or without epinephrine mixed with 20 ccs mixed with 20 ccs bupivacaine hydrochloride

2 ccs methylprednisolone acetate sterile solution—40 mg/cc

1 cc dexamenthasone phosphate/4 mg/cc

2 cc absolute alcohol

50 ccs—total

GLAL solution #9

20 ccs 2% lidocaine hydrochloride with or without epinephrine mixed with 20 ccs bupivacaine hydrochloride

2 ccs methylprednisolone acetate sterile solution—80 mg/cc

1 cc dexamenthasone phosphate/4 mg/cc

7 ccs dehydrated alcohol

50 ccs—total

What is claimed is:

1. A medication comprising about 45 ccs of 0.5% bupivacaine hydrochloride, about 2 ccs of methylprednisolone acetate sterile aqueous suspension 80 mg/cc; about 1 cc of Dexamethasone Sodium Phosphate 4 mg/cc; and about 2 ccs of 1% absolute alcohol for a total of about 50 ccs of medication.

2. A medication according to claim 1 further comprising epinephrine.

3. A method of medically treating a neuropathology, comprising the steps of injecting about 1 cc of a medication into an inter metatarsal, neuropathological space, the medication comprising about 45 ccs of 0.5% bupivacaine hydrochloride; about 2 ccs of methylprednisolone acetate sterile aqueous suspension 80 mg/cc; about 1 cc Dexamethosone Sodium Phosphate 4 mg/cc; and about 2 cc of 1% absolute alcohol.

4. A method according to claim 3 including the steps of repeating the injections every two weeks.

5. A method according to claim 3 including the steps of repeating the injections three times for a total of four injections.

6. A medication comprising about 45 ccs of one or both of the materials selected from the group consisting of bupivacaine hydrochloride and lidocaine hydrochloride; about 2 ccs of methylprednisolone acetate sterile aqueous suspension 80 mg/cc; about icc of Dexamethosone Sodium Phosphate 4 mg/cc; and about 2 ccs of 1% absolute alcohol.

7. A medication according to claim 6 further including epinephrine.

8. A medication comprising about 90% by volume of 0.5% bupivacaine hydrochloride, about 4% by volume of methylprednisolone acetate sterile aqueous suspension 80 mg/cc; about 2% by volume of Dexamethasone Sodium Phosphate 4 mg/cc; and about 4% by volume of 1% absolute alcohol.

9. A medication according to claim 8 further comprising epinephrine.

10. A method of medically treating a neuropathology, comprising the steps of injecting about icc of a medication into an inter metatarsal, neuropathological space, the medication comprising about 90% by volume of 0.5% bupivacaine hydrochloride; about 4% by volume of methylprednisolone acetate sterile aqueous suspension 80 mg/cc; about 2% by volume Dexamethosone Sodium Phosphate 4 mg/cc; and about 4% by volume of 1% absolute alcohol.

11. A method according to claim 10 including the steps of repeating the injections every two weeks.

12. A method according to claim 10 including the steps of repeating the injections three times for a total of four injections.

13. A medication comprising about 90% by volume of one or both of the materials selected from the group consisting of bupivacaine hydrochloride and lidocaine hydrochloride; about 4% by volume of methylprednisolone acetate sterile aqueous suspension 80 mg/cc; about 2% by volume of Dexamethosone Sodium Phosphate 4 mg/cc; and about 4% by volume of 1% absolute alcohol.

14. A medication according to claim 6 further including epinephrine.
